(19) United States
(12) Patent Application Publication
(10) Pub. No.: US 2024/0074942 A1
(43) Pub. Date: Mar. 7, 2024

(54) HUMAN ECOLOGICAL NEGATIVE OXYGEN ION CELL ACTIVATION AND MERIDIAN MOXIBUSTION INTELLIGENT COMMUNICATION PHYSICAL THERAPY PLATFORM

(71) Applicant: Zhejiang Cloud Moxibustion Biotechnology Co., Ltd, Ningbo (CN)

(72) Inventors: Junqun TAN, Ningbo (CN); Peijun LIU, Ningbo (CN); Xiao TAN, Ningbo (CN)

(21) Appl. No.: 18/092,706

(22) Filed: Jan. 3, 2023

(30) Foreign Application Priority Data
Sep. 2, 2022 (CN) 202211072008X

(51) Int. Cl. A61H 39/06; A61G 13/00

(52) U.S. Cl. CPC A61H 39/06; A61G 13/009; A61G 2200/327; A61G 2203/20; A61G 2203/70; A61H 2201/105

(57) ABSTRACT

A human ecological negative oxygen ion cell activation and meridian moxibustion intelligent communication physical therapy platform, including a base plate, lifting assembly and smoke discharging assembly, wherein the lifting assembly is installed at a front surface of the base plate, the smoke discharging assembly is installed inside the base plate and the lifting assembly, and the lifting assembly includes a physical therapy box, sliding rails, threaded rods, a motor, a sliding block and a square block; a C-shaped stander is installed at the front surface of the base plate, two fixing plates are symmetrically installed at an inner surface of the C-shaped stander, at least one installation shielding plate is installed at neighboring sides of the fixing plates, a front surface of the at least one installation shielding plate is installed at outer sides of the sliding rails, the sliding rails are slidably connected with the sliding block.

### TECHNICAL FIELD

The present invention relates to the technical field of medical and health care apparatus, especially to a human ecological negative oxygen ion cell activation and meridian moxibustion intelligent communication physical therapy platform.

### BACKGROUND TECHNOLOGY

Modern medical researches revealed that, occurrence processes of suboptimal health conditions are processes of loss of balance of the immune system. After the imbalance of the immune system, functions of the immune system decline, compositions and activities of the immune cells become low, cell treatment is to improve the quantity of the immune cells with physical methods of moxibustion, promote differentiation of the immune cells, supplement the immune cells in full aspects by amplification in vitro and inducement of autoimmune cells, maintain a high level of quantity of the immune cells so as to adjust the immune system, improve physical immunity, enhance physical conditions and protect the human body from the inside out;

For the moxibustion devices currently available, as the moxibustion height is fixed, it is not possible to adjust the height, the utility rate of the Moxa cones is low, during moxibustion, without assistance of negative oxygen ions, moxibustion is done to the entire body of the subject while the effect is not satisfactory, when making moxibustion, as there is no foreign human examination device, it is not possible to collect information on the human body in real time, specific health conditions are unknown, furthermore, during moxibustion, a lot of fogs are formed, which fill the house, user experience is thus poor, therefore, a human ecological negative oxygen ion cell activation and meridian moxibustion intelligent communication platform is proposed.

### SUMMARY OF THE INVENTION

In view of the above conditions, the present invention aims to provide a human ecological negative oxygen ion cell activation and meridian moxibustion intelligent communication platform, to address or relieve the technical problems existing in the prior art, or at least provide an advantageous choice.

Technical solutions of the present invention are realized in this way: a human ecological negative oxygen ion cell activation and meridian moxibustion intelligent communication physical therapy platform, comprising a base plate, a lifting assembly and a smoke discharging assembly, wherein the lifting assembly is installed at a front surface of the base plate, the smoke discharging assembly is installed inside the base plate and the lifting assembly, and the lifting assembly comprises a physical therapy box, sliding rails, threaded rods, a motor, a sliding block and a square block;

A C-shaped stander is installed at the front surface of the base plate, two fixing plates are symmetrically installed at an inner surface of the C-shaped stander, at least one installation shielding plate is installed at neighboring sides of the fixing plates, a front surface of the at least one installation shielding plate is installed at outer sides of the sliding rails, the sliding rails are slidably connected with the sliding block, an inner top surface of the at least one installation shielding plate is connected with top ends of the threaded rods rotatably by at least one bearing, a lower surface of the at least one installation shielding plate is installed at an upper surface of the motor, an output shaft of the motor penetrates through the lower surface of the at least one installation shielding plate and is welded to bottom ends of the threaded rods, an inner surface of the sliding block is welded to an outer surface of the square block, threaded through-holes are provided at an upper surface of the square block, inner surfaces of the threaded through-holes are connected with outer surfaces of the threaded rods via threads, a smoke collection box is installed at a rear surface of the sliding block, three smoke vent holes are symmetrically provided at a front surface of the physical therapy box, a side of the smoke collection box is installed at one of the smoke exhaust holes at the front surface of the physical therapy box, moxibustion boxes are uniformly installed in the physical therapy box, medical ecological negative oxygen ion generators are uniformly installed at an inner surface of the physical therapy box, a dynamic human biological information analyzer, a data processor and a display are respectively installed at a side of the base plate.

Further preferably, the smoke discharging assembly comprises first smoke absorption ducts, a second smoke absorption duct, a third smoke absorption duct and a metallic active catalytic moxa smoke processor;

The first smoke absorption ducts are configured to be two, the first smoke absorption ducts are installed respectively in two of the smoke exhaust holes, neighboring ends of the first smoke absorption ducts penetrate through an outer surface of the smoke collection box, two fourth smoke absorption boxes are provided in the physical therapy box, ends of the fourth smoke absorption ducts are installed at the smoke exhaust holes in the physical therapy box, a top end of the second smoke absorption duct penetrates through a lower surface of the smoke collection box, an outer surface of the second smoke absorption duct is attached to an inner surface of the third smoke absorption duct, an end of the third smoke absorption duct penetrates through the front surface of the base plate and is installed at a smoke inlet of the metal active catalytic moxa smoke processor, and the base plate is installed outside the metal active catalytic moxa smoke processor.

Further preferably, a physical therapy bed is installed at an upper surface of the base plate, thermal dissemination holes are provided evenly at an outer surface of the base plate, the metal ecological negative oxygen ion generator comprises a negative ion generator, an upper cover plate, an output conduction wire, a radiation circuit board, a welding portion, electric discharging points and a lower cover plate;

The negative ion generator is installed at a side of the upper cover plate far from the radiation circuit board, the upper cover plate, the radiation circuit board and the lower cover plate are fixed at the inner surface of the physical therapy box via bolts, an electric output end of the negative ion generator is electrically connected with an end of the output conduction wire, a copper covered region is provided at a side of the radiation circuit board far from the upper cover plate, the welding portion is provided at the copper covered region, another end of the output conduction wire passes sequentially the upper cover plate and a side of the radiation circuit board and is electrically connected with a rear surface of the welding portion and the electric discharging points are installed evenly at a front surface of the copper covered region.

Further preferably, a printing device and a keyboard are respectively installed at a side of the base plate.

Further preferably, at least one biological electric data collection oversleeve is installed evenly at the upper surface of the physical therapy bed, and a multi-point human biological signal collector is installed at the upper surface of the physical therapy bed.

Further preferably, at least one smoke discharging duct is installed at smoke outlets of the metal active catalytic moxa smoke processor, and an end of the at least one smoke discharging duct penetrates through the base plate.

Further preferably, at least one movable through-slot is provided at a rear surface of the at least one installation shielding plate, an inner surface of the at least one movable through-slot is attached to an outer surface of the smoke collection box.

Further preferably, supporting legs are provided at the front surface of the base plate.

Further preferably, at least one stopping block is provided outside the threaded rods, and at least one protection block is installed at a top end of the third smoke absorption duct.

Further preferably, the dynamic human biological information analyzer is configured to detect and analyze real time changes of biological indicators of a subject, and generate a comprehensive examination report, wherein the data processor is configured to realize data storage, backup examination and analyze data of the subject, track historical data, and can be connected with instant communication software, and also by extension functions of the data processor communication of health detection information of the subject with other special medical information platform can be done.

By providing the foregoing technical solutions, the present invention has the following beneficial effects:
1. In the present invention, by configuring the lifting assembly, it is convenient to select a moxibustion height suitable for the subject depending on individual differences, so as to promise the optimum use height and improve the utility rate of ecological negative oxygen ions and energies in the moxa cone and good moxabustion effects are achieved.
2. In the present invention, by the high concentration negative oxygen ions generated by the medical ecological negative oxygen ion generator and the far infrared rays generated by the moxa cones in the moxa box, moxibustion is given to the entire body of the subject and significant effects are achieved.
3. In the present invention, by analyzing with the dynamic human biological information analyzer the real-time human information collected with the at at least one biological electric data collection oversleeve and the plurality of human biological signal collectors, transmitting with the dynamic human biological information analyzer the analyzed data to the data processor, transmitting the data in the data processor to the display and printing with the printing device the report by operating on the keyboard, during conducting physical therapy cell and vital energy and blood improvement effects can be monitored in real time, and real-time imaging report can be generated, and by the data processor network communication and remote control with immediate communication software can be realized and it is also possible for the subject to view in real time current human vital energy and blood indicators and parameters with instant communication software.
4. In the present invention, by the use of the smoke discharging assembly, it is convenient to filter the smoke generated during moxibustion until the smoke is qualified for discharging according to environment protection standards and discharge the same, in this way, influences of the smoke generated during moxibustion on the environment and the human bodies can be reduced and moxibustion comfort can be improved.

The foregoing description is only given for the sake of clarity and is not intended to limit the present invention in any sense. Except the aspects, embodiments and features described in the foregoing paragraphs, by referring to the accompanying drawings and the following detailed description, further aspects, embodiments and features of the present invention are obvious.

### BRIEF DESCRIPTION OF DRAWINGS

In order to explain the technical solutions in embodiments of the present invention or the prior art, hereinafter a simple introduction will be given to the accompanying drawings to be used in the description of the embodiments and the prior art, obviously, the accompanying drawings show only some of the embodiments of the present invention, and for those of ordinary skill in the art, without paying creative effort, it is still possible to obtain other drawings on the basis of these drawings.

FIG. 1 is a structural diagram showing the present invention;

FIG. 2 is a structural diagram of the present invention taken from a rear side;

FIG. 3 is a sectional structural diagram taken along A-A of FIG. 2 in the present invention;

FIG. 4 is an enlarged structural diagram showing a region B in FIG. 3 of the present invention;

FIG. 5 is a sectional structural diagram taken along C-C of FIG. 3 of the present invention;

FIG. 6 is an enlarged structural diagram showing a region D in FIG. 5 of the present invention;

FIG. 7 is an enlarged structural diagram showing a region E in FIG. 5 of the present invention;

FIG. 8 is an internal structural diagram showing installation of the base plate and the metal active catalytic moxa smoke processor in the present invention;

FIG. 9 is an isometric structural diagram showing the present invention viewing from a top side; and

FIG. 10 is a structural diagram showing a detached medical ecological negative oxygen ion generator in the present invention.

Markups in the drawings: 1, base plate; 2, lifting assembly; 201, physical therapy box; 202, sliding rail; 203, threaded rod; 204, motor; 205, sliding block; 206, square block; 3, smoke discharging assembly; 301, first smoke absorption duct; 302, second smoke absorption duct; 303, third smoke absorption duct; 304, metal active catalytic moxa smoke processor; 305, fourth smoke absorption duct; 4, fixing plate; 5, C-shaped stander; 6, smoke collection box; 7, physical therapy bed; 8, biological electric data collection oversleeve; 9, supporting leg; 10, multi-point human biological signal collector; 11, dynamic human biological information analyzer; 12, data processor; 13, printing device; 14, keyboard; 15, display; 16, installation shielding plate; 17, movable through-slot; 18, medical ecological negative oxygen ion generator; 19, moxa cone box; 21, thermal dissemination hole; 22, stopping block; 23, protection block; 24, threaded through-hole; 25, smoke discharging duct; 26, negative ion generator; 27, upper cover plate; 28, output conduction wire; 29, radiation circuit board; 30, copper covered region; 31, welding portion; 32, electric discharging point; and 33 lower cover plate.

### EMBODIMENTS

In the following paragraphs, only some exemplary embodiments have been simply described. As those skilled in the art know, without departing from the spirit and the scope of the present invention, it is possible to modify the given embodiment in a variety of ways. Therefore, the accompanying drawings and the description shall be taken as exemplary rather than restrictive.

Hereinafter a detailed description will be given to the embodiments of the present invention in conjunction with the accompanying drawings.

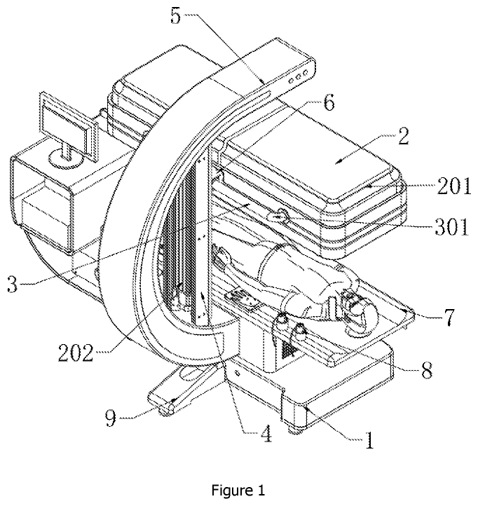

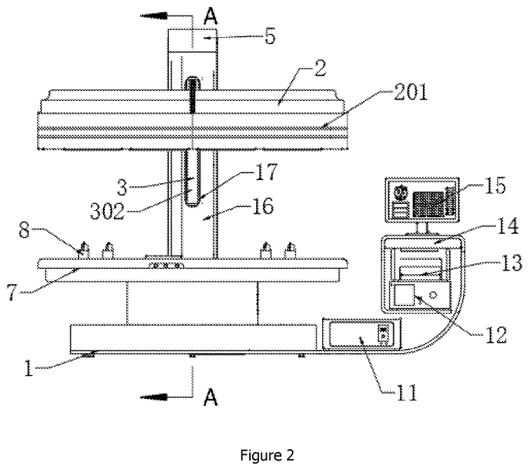

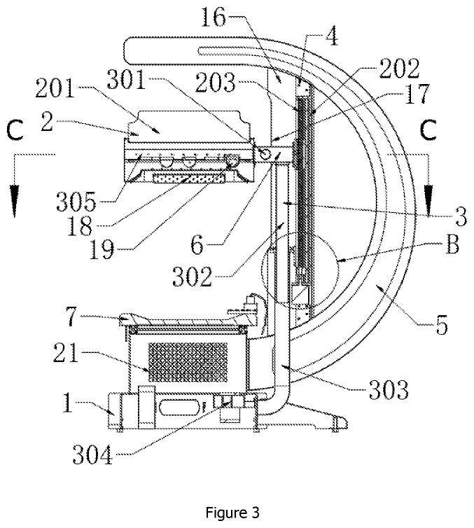

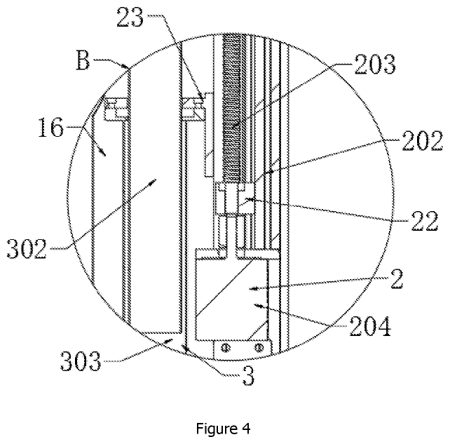

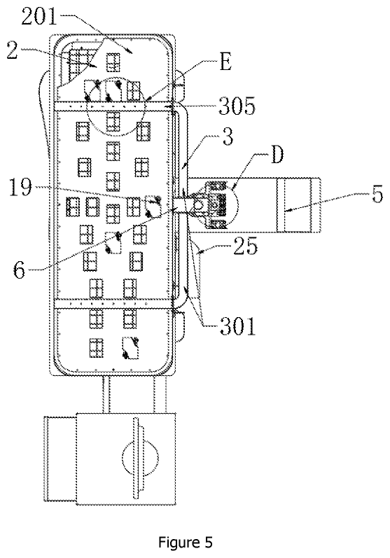

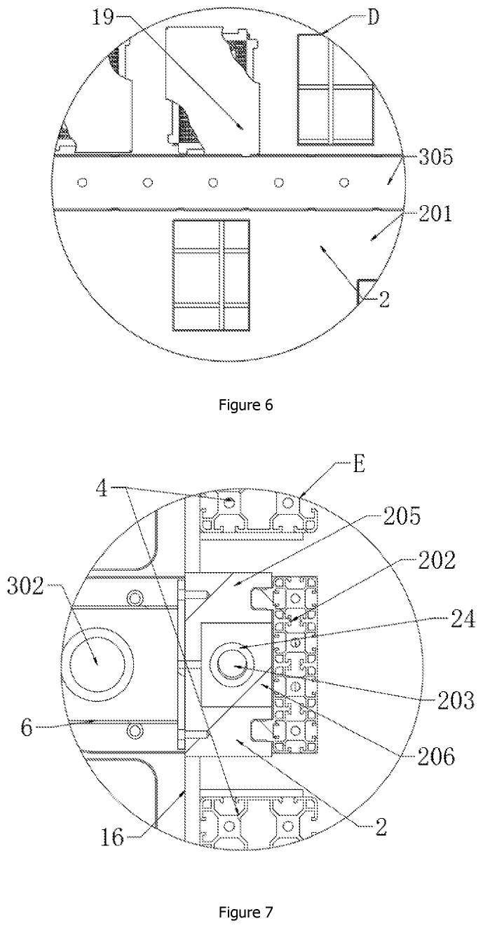

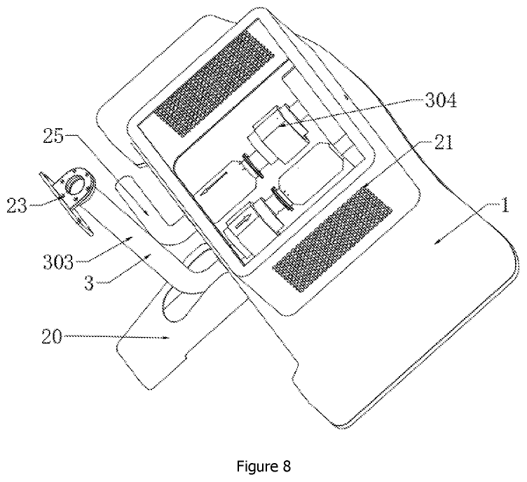

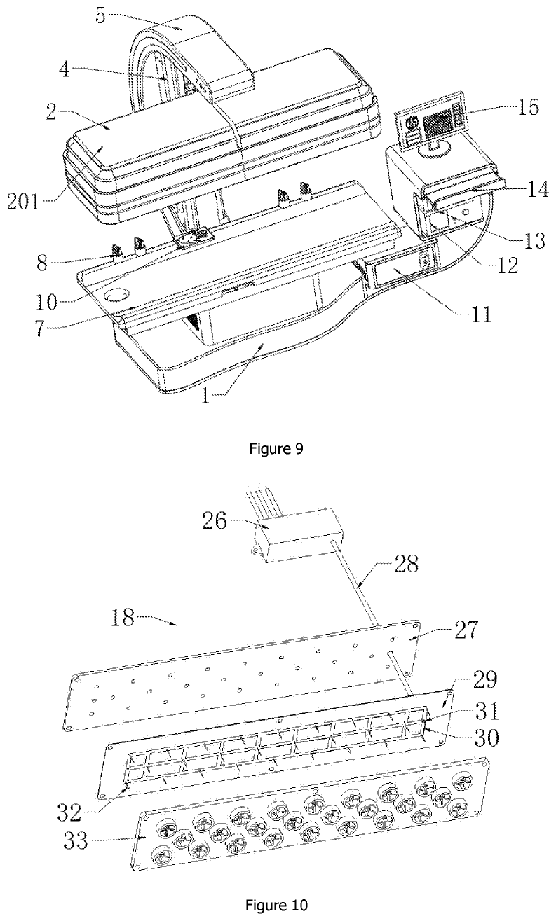

As shown in FIGS. 1-10, embodiments of the present invention provide a human ecological negative oxygen ion cell activation and meridian moxibustion intelligent communication physical therapy platform, comprises a base plate 1, a lifting assembly 2 and an smoke discharging assembly 3, the lifting assembly 2 is installed at a front surface of the base plate 1, the smoke discharging assembly 3 is installed in the base plate 1 and the lifting assembly 2, the lifting assembly 2 comprises a physical therapy box 201, sliding rails 202, threaded rods 203, a motor 204, a sliding block 205 and a square block 206;

A C-shaped stander 5 is installed at the front surface of the base plate 1, two fixing plates 4 are installed at an inner surface of the C-shaped stander 5, an installation shielding plate 16 is installed at a side of the fixing plates 4, a front surface of the installation shielding plate 16 is installed at outer surfaces of the sliding rails 202, the sliding rails 202 are slidably connected with the sliding block 205, an inner top end of the installation shielding plate 16 is connected rotatably with top ends of the threaded rods 203, a lower surface of the installation shielding plate 16 is installed on an upper surface of the motor 204, an output shaft of the motor 204 penetrates through the lower surface of the installation shielding plate 16 and is welded at bottom ends of the threaded rods 203, an inner side of the sliding block 205 is welded at an outer surface of the square block 206, threaded through-holes 24 are provided at an upper surface of the square block 206, inner surfaces of the threaded through-holes 24 are connected with outer surfaces of the threaded rods 204, a smoke collection box 6 is installed at a rear surface of the sliding block 205, three smoke vent holes are symmetrically provided at a front surface of the physical therapy box 201, a side of the smoke collection box 6 is installed at one of the smoke vent holes at the front surface of the physical therapy box 201, moxa cone boxes 19 are evenly installed in the physical therapy box 201, medical ecological negative oxygen ion radiators 8 are installed uniformly at an inner surface of the physical therapy box 201, a dynamic human biological information analyzer 11, a data processor 12 and a display 15 are installed respectively at a side of the base plate 1.

In an embodiment, the smoke discharging assembly 3 comprises first smoke absorption ducts 301, a second smoke absorption duct 302, a third smoke absorption duct 303 and a metal active catalytic moxibustion smoke processor 304;

The first smoke absorption ducts 301 are configured to be two, ends of the two first smoke absorption ducts 301 are installed in the smoke vent holes at the front surface of the physical therapeutic box 201, another ends of the two first smoke absorption ducts 301 penetrate through an outer surface of the smoke collection box 6, two fourth smoke absorption ducts 305 are installed in the physical therapy box 201, ends of the two fourth smoke absorption ducts 305 are installed in two of the smoke vent holes in the physical therapy box 201, a lower surface of the smoke collection box 6 passes through a top end of the second smoke absorption duct 302, an outer surface of the second smoke absorption duct 302 is attached to an inner surface of the third smoke absorption duct 303, an end of the third smoke absorption duct 303 penetrates through the front surface of the base plate 1, and is installed at a smoke inlet of the metal active catalytic moxa smoke processor 304, the metal active catalytic moxa smoke processor 304 is installed inside the base plate 1, by starting the metal active catalytic moxa smoke processor 304, the metal active catalytic moxa smoke processor 304 draws the smoke generated during combustion of the moxa cones in the physical therapy box 201 via sequentially the third smoke absorption duct 303, the second smoke absorption duct 302, the smoke collection box 6, the first smoke absorption ducts 301 and the fourth smoke absorption ducts 305 into the metal active catalytic moxa smoke processor 304.

In an embodiment, a physical therapy bed 7 is installed at an upper surface of the base plate 1, thermal dissemination holes 21 are provided uniformly at the outer surface of the base plate 1, the medical ecological negative oxygen ion generator 18 comprises a negative ion generator 26, an upper cover plate 27, an output conduction wire 28, a radiation circuit board 29, a welding portion 31, electric discharging points 32 and a lower cover plate 33;

The negative ion radiator 26 is installed at a side of the upper cover plate 27 far from the radiation circuit board 29, the upper cover plate 27, the radiation circuit board 29 and the lower cover plate 33 are fixed at the inner surface of the physical therapy box 201, an electric output end of the negative ion generator 26 is electrically connected to an end of the output conduction wire 28, a copper covered region 30 is installed at a side of the radiation circuit board 29 far from the upper cover plate 27, the welding portion 31 is provided on the copper covered region 30, another end of the output conduction wire 28 passes sequentially through the upper cover plate 27 and a side of the radiation circuit board 29 and is electrically connected with a rear surface of the welding portion 31, electric discharging points 32 are installed uniformly at a front surface of the copper covered region 30, and by configuration of the physical therapy bed 7, the subject can lie on top thereof, usage comfort is improved, wherein, by configuration of the thermal dissemination holes 21, heat generated due to work of the metal active catalytic moxa smoke processor 34 is disseminated, wherein a pulse oscillation circuit passes through the negative ion generator 26 in the medical ecological negative oxygen ion generator 18, low voltage when passing the negative ion generator 26 is converted to be DC negative high voltage, passes the output conduction wire 28 and is output to the radiation circuit board 29, tips of the electric discharging points 32 installed on the copper covered region 30 generate negative high corona.

In an embodiment, a printing device 13 and a keyboard 14 are installed respectively at a side of the base plate 1, by configuration of the printing device 13, it is convenient to print the report for the subject, wherein configuration of the keyboard 14 makes it convenient to operate the display 15.

In an embodiment, biological electric data collection oversleeves 8 are installed evenly at the upper surface of the physical therapy bed 7, a multi-point human biological signal collector 10 is installed at the upper surface of the physical therapeutic bed 7, and real-time human information collected by the biological electric data collection oversleeves 8 and the multi-point human biological signal collector 10 is transmitted to the dynamic human biological information analyzer 11 for analysis.

In an embodiment, a smoke discharging duct 25 is installed at a smoke outlet port of the metal active catalytic moxa smoke processor 304, an end of the smoke discharging duct 25 penetrates through the base plate 1, and by configuration of the smoke discharging duct 25, the smoke is processed by the metal active catalytic moxa smoke processor 30 and discharged via the smoke discharging duct 25.

In an embodiment, a movable through-slot 17 is provided at a rear surface of the installation shielding plate 16, an inner surface of the movable through-slot 17 is attached to an outer surface of the smoke collection box 16, and by configuration of the movable through-slot 17 it is convenient to move the smoke collection box 6.

In an embodiment, supporting legs 9 are provided at the front surface of the base plate 1, and by configuration of the supporting legs 9 the base plate 1 is more stable.

In an embodiment, a stopping block 22 is provided outside the threaded rods 203, a protection block 23 is installed at a top end of the third smoke absorption duct 303, by configuration of the stopping block 22, falling off of the square block 206 from the threaded rods 203 is efficiently prevented and by configuration of the protection block 23, a moving position of the smoke collection box 6 can be effectively limited.

In an embodiment, the dynamic human biological information analyzer 11 can detect and analyze real-time changes of physical indicators of the subject and generate a comprehensive examination report, the data processor 12 can realize data storage, inspection and analysis data backup, historical data tracking and can also be connected with foreign communication software, and extension functions of the data processor 12 can also enable interchange of health inspection information of the subject with other special medical information platforms.

In an embodiment, openings are provided at outer surfaces of the fourth smoke absorption ducts 305, so that it is convenient to draw the smoke generated due to moxibustion.

In an embodiment, an electric output end of the display 15 is respectively electrically connected with an electric input end of the motor 204, the metal active catalytic moxa smoke processor 305, the biological electrical data collection oversleeves 8, the multi-point human biological signal collector 10 and the medical ecological negative oxygen ion generator 18, signal output ends of the biological electric data collection oversleeves 8 and the multi-point human biological signal collector 10 are connected electrically via conduction wires to a signal input end of the dynamic human biological information analyzer 11, a signal output end of the dynamic human biological information analyzer 11 is connected electrically via conduction wires to a signal input end of the data processor 12, and a signal output end of the data processor 12 is connected electrically to a signal input end of the display 15 via conduction wires.

In an embodiment, a switch for controlling on and off of the display 15, the dynamic human biological information analyzer 11, the data processor 12 and the printing device 13 is installed at a side of the base plate, the switch is electrically connected with foreign power supply, and is configured to supply energy to the display 15, the dynamic human biological information analyzer 11, the data processor 12 and the printing device 13.

In an embodiment, an electrical input end of the negative ion generator 26 is respectively connected with grounding electrical wires and 12V positive and negative power supplies.

During operation of the present invention, when the subject lies on the physical therapy bed 7, a PLC controller is provided in the display 15, the display 15 is a touch screen and is electrically connected with the keyboard 14, so that it is convenient to operate the display 15 and the keyboard 14 to start or shut down the motor 204, the metal active catalytic moxa smoke processor 304, the biological electric data collection oversleeves 8, the multi-point human biological signal collector 10 and the medical ecological negative oxygen ion generator 18, the pulse oscillation circuit passes through the negative ion generator 26 in the medical ecological negative oxygen ion generator 18, low voltage is boosted by a high voltage module in the negative ion generator 26 to be DC negative high voltage, the high voltage is output via the output conduction wires 28 to the radiation circuit board 29, and tips of the electric discharging points 32 installed on the copper covered region 30 generate negative DC high corona, a large quantity of electrons are radiated at a high speed, the electrons cannot exist in the air for a long time and unite with oxygen molecules in the air immediately, so that negative oxygen ions are continuously output, at this time, the PLC controller in the display 15 controls the motor 204 to rotate, the output shaft of the motor 204 drives the threaded rods 203 to rotate in the threaded through-holes 24 on the square block 206, so that the sliding block 205 moves up and down on the sliding rails 202, the sliding block 205 drives the smoke collection box 6 to move up and down, the smoke collection box 6 in the meanwhile drives the second smoke absorption duct 302 and the physical therapy box 201 to move to above the subject, at this time, put moxa cones into the moxa cone box 19, the moxa cone box 19 is distributed reasonably according to primary meridian principles of the human body, a lot of far infrared lights and heat generated by moxibustion can realize overall gentle conditioning of the human body, start the biological electric data collection oversleeves 8, the multi-point human biological signal collector 10 and the medical ecological negative oxygen ion generator 18, fix the biological electric data collection oversleeves 8 on the subject, place a hand of the subject on the multi-point biological signal collector 10, real-time human information collected via the biological electric data collection oversleeves 8 and the multi-point human biological signal collector 10 is transmitted to the dynamic human biological information analyzer 11 for analysis, thereafter, the dynamic human biological information analyzer 11 transmits the data to the data processor 12, the data processor 12 in turn transmits the data in the data processor 12 to the display 15, by operating the keyboard 14 the report can be printed via the printing device 13, in this way, cell and vital energy and blood conditioning effects during physical therapeutic treatment can be monitored in real time and a real-time imaging report can be generated, furthermore, by the data processor it is possible to connected with communication software for network interconnection and remote control, the subject can also view current vital energy and blood indicators on the communication software installed on a cellphone, during moxibustion, start the metal active catalytic moxa smoke processor 304, the metal active catalytic moxa smoke processor 304 draws the smoke generated due to moxibustion in the physical therapy box 201 via the third smoke absorption duct 303, the second smoke absorption duct 302, the smoke collection box 6, the first smoke absorption ducts 301 and the fourth smoke absorption ducts 305 and after treatment at the metal active catalytic moxa smoke processor the smoke is discharged via the smoke discharging duct 25.

The foregoing are only some specific embodiments of the present invention, however, the protection scope of the present invention is not limited to the embodiments disclosed herein, any one skilled in the art can easily come up with all kinds of changes or replacement within the technical scope disclosed in the present invention and all the changes and replacements are covered in the protection scope of the present invention. Therefore, the protection scope of the present invention shall be defined by the protection scope of the appended claims.

1. A human ecological negative oxygen ion cell activation and meridian moxibustion intelligent communication physical therapy platform, comprising a base plate, a lifting assembly and a smoke discharging assembly, wherein the lifting assembly is installed at a front surface of the base plate, the smoke discharging assembly is installed inside the base plate and the lifting assembly, and the lifting assembly comprises a physical therapy box, sliding rails, threaded rods, a motor, a sliding block and a square block;
a C-shaped stander is installed at the front surface of the base plate, two fixing plates are symmetrically installed at an inner surface of the C-shaped stander, at least one installation shielding plate is installed at neighboring sides of the fixing plates, a front surface of the at least one installation shielding plate is installed at outer sides of the sliding rails, the sliding rails are slidably connected with the sliding block, an inner top surface of the at least one installation shielding plate is connected with top ends of the threaded rods rotatably by at least one bearing, a lower surface of the at least one installation shielding plate is installed at an upper surface of the motor, an output shaft of the motor penetrates through the lower surface of the at least one installation shielding plate and is welded to bottom ends of the threaded rods, an inner surface of the sliding block is welded to an outer surface of the square block, threaded through-holes are provided at an upper surface of the square block, inner surfaces of the threaded through-holes are connected with outer surfaces of the threaded rods via threads, a smoke collection box is installed at a rear surface of the sliding block, three smoke vent holes are symmetrically provided at a front surface of the physical therapy box, a side of the smoke collection box is installed at one of the smoke exhaust holes at the front surface of the physical therapy box, moxibustion boxes are uniformly installed in the physical therapy box, medical ecological negative oxygen ion generators are uniformly installed at an inner surface of the physical therapy box, a dynamic human biological information analyzer, a data processor and a display are respectively installed at a side of the base plate.

2. The human ecological negative oxygen ion cell activation and meridian moxibustion intelligent communication physical therapy platform according to claim 1, wherein the smoke discharging assembly comprises first smoke absorption ducts, a second smoke absorption duct, a third smoke absorption duct and a metallic active catalytic moxa smoke processor;
the first smoke absorption ducts are configured to be two, the first smoke absorption ducts are installed respectively in two of the smoke exhaust holes, neighboring ends of the first smoke absorption ducts penetrate through an outer surface of the smoke collection box, two fourth smoke absorption boxes are provided in the physical therapy box, ends of the fourth smoke absorption ducts are installed at the smoke exhaust holes in the physical therapy box, a top end of the second smoke absorption duct penetrates through a lower surface of the smoke collection box, an outer surface of the second smoke absorption duct is attached to an inner surface of the third smoke absorption duct, an end of the third smoke absorption duct penetrates through the front surface of the base plate and is installed at a smoke inlet of the metal active catalytic moxa smoke processor, and the base plate is installed outside the metal active catalytic moxa smoke processor.

3. The human ecological negative oxygen ion cell activation and meridian moxibustion intelligent communication physical therapy platform according to claim 1, wherein a physical therapy bed is installed at an upper surface of the base plate, thermal dissemination holes are provided evenly at an outer surface of the base plate, the metal ecological negative oxygen ion generator comprises a negative ion generator, an upper cover plate, an output conduction wire, a radiation circuit board, a welding portion, electric discharging points and a lower cover plate;
the negative ion generator is installed at a side of the upper cover plate far from the radiation circuit board, the upper cover plate, the radiation circuit board and the lower cover plate are fixed at the inner surface of the physical therapy box via bolts, an electric output end of the negative ion generator is electrically connected with an end of the output conduction wire, a copper covered region is provided at a side of the radiation circuit board far from the upper cover plate, the welding portion is provided at the copper covered region, another end of the output conduction wire passes sequentially the upper cover plate and a side of the radiation circuit board and is electrically connected with a rear surface of the welding portion and the electric discharging points are installed evenly at a front surface of the copper covered region.

4. The human ecological negative oxygen ion cell activation and meridian moxibustion intelligent communication physical therapy platform according to claim 1, wherein a printing device and a keyboard are respectively installed at a side of the base plate.

5. The human ecological negative oxygen ion cell activation and meridian moxibustion intelligent communication physical therapy platform according to claim 3, wherein at least one biological electric data collection oversleeve is installed evenly at the upper surface of the physical therapy bed, and a multi-point human biological signal collector is installed at the upper surface of the physical therapy bed.

6. The human ecological negative oxygen ion cell activation and meridian moxibustion intelligent communication physical therapy platform according to claim 2, wherein at least one smoke discharging duct is installed at smoke outlets of the metal active catalytic moxa smoke processor, and an end of the at least one smoke discharging duct penetrates through the base plate.

7. The human ecological negative oxygen ion cell activation and meridian moxibustion intelligent communication physical therapy platform according to claim 1, wherein at least one movable through-slot is provided at a rear surface of the at least one installation shielding plate, an inner surface of the at least one movable through-slot is attached to an outer surface of the smoke collection box.

8. The human ecological negative oxygen ion cell activation and meridian moxibustion intelligent communication physical therapy platform according to claim 1, wherein supporting legs are provided at the front surface of the base plate.

9. The human ecological negative oxygen ion cell activation and meridian moxibustion intelligent communication physical therapy platform according to claim 2, wherein at least one stopping block is provided outside the threaded rods, and at least one protection block is installed at a top end of the third smoke absorption duct.

10. The human ecological negative oxygen ion cell activation and meridian moxibustion intelligent communication physical therapy platform according to claim 1, wherein the dynamic human biological information analyzer is configured to detect and analyze real time changes of biological indicators of a subject, and generate a comprehensive examination report, wherein the data processor is configured to realize data storage, backup examination and analyze data of the subject, track historical data, and can be connected with instant communication software, and also by extension functions of the data processor communication of health detection information of the subject with other special medical information platform can be done.
